(19) United States
(12) Patent Application Publication
(10) Pub. No.: US 2012/0123292 A1
(43) Pub. Date: May 17, 2012

(54) Nerve Mapping Surgical System and Method of Use of Dual Function Surgical Instrument Within Such System

(75) Inventors: Randy Fagin, Austin, TX (US); Jon Schiff, Austin, TX (US); Jann Bonfils-Rasmussen, Leander, TX (US)

(73) Assignee: ProPep Surgical, LLC, Austin, TX (US)

(21) Appl. No.: 13/107,855

(22) Filed: May 13, 2011

Related U.S. Application Data
(63) Continuation-in-part of application No. 11/745,505, filed on May 8, 2007, now Pat. No. 8,083,685.

(51) Int. Cl. A61B 5/05; A61B 18/18; A61B 19/00

(52) U.S. Cl. 600/554; 606/129; 606/41

(57) ABSTRACT

A surgical system has an electrode probe, an analyzer, a bipolar instrument, a bipolar power supply and a switch, the switch in communication with the analyzer, the bipolar power supply and the bipolar instrument, to permit alternate use of the bipolar instrument as a surgical instrument and use as an exploratory probe. An electrode probe delivery device has a base plate, an insertion guide connected to and extending from the base plate and formed for receipt of an electrode probe. A catheter on the device permits receipt of at least one electrode probe and a handle connects to the catheter and permits movement thereof such that the catheter is passable through the base plate for introduction into a patient.

### CROSS REFERENCE TO RELATED APPLICATION

The present application is a continuation-in-part of and relies upon and claims the benefit of the filing date of pending application Ser. No. 11/745,505. System and Method for Laparoscopic Nerve Detection, filed May 8, 2007.

### BACKGROUND OF THE INVENTION

1. Field of the Invention

The present invention relates in general to the field of laparoscopic surgery, and more particularly, to a surgical system and method of use of a dual function surgical probe within the system to facilitate detection and mapping of nerve locations.

2. Description of the Related Art

Traditionally, surgery on internal body parts was performed by cutting an incision in the skin to access the internal body parts. Such open surgery entails a number of known risks including infection, inadvertent damage to other organs and structures, scarring, and loss of blood. In an effort to reduce some of these risks and improve patient outcomes surgeons have developed laparoscopic, and more recently robotic, techniques to perform surgery. Robotic surgery is essentially an advanced type of laparoscopic surgery in which the arms that enter the body cavity are robotically controlled instead of manually controlled. During a laparoscopic or robotic surgery, small incisions are made in the skin through which 5-12 millimeter access ports are placed. These ports serve as doorways through which small working instruments and a camera can be placed. The camera creates a magnified view of the internal organs that the surgeon sees on a monitor or console. Such less invasive laparoscopic and robotic surgeries typically have reduced side effects for the patient to allow a more rapid and complete recovery.

One example where laparoscopic and robotic surgery has gained acceptance with positive results is for the accomplishment of a radical prostatectomy. Conventionally, a radical prostatectomy is performed by cutting an incision at the base of the pelvic bone to gain access to the prostate. Once visible, the prostate is cut from the surrounding tissue and removed. Because the area around the prostate is rich in nerves and muscles that support sexual and urinary functions, a radical prostatectomy can cause severe side effects, including sexual dysfunction and incontinence. For example, up to half of conventionally-performed radical prostatectomies result in permanent erectile dysfunction. In contrast, a radical laparoscopic or robotic prostatectomy has the potential for far fewer side effects. In part, laparoscopic and robotic prostatectomies tend to have fewer side effects because the procedure affords the surgeon improved vision and in the case of robotics in particular, more dexterous tools as well. In the case of robotic surgery the improved vision and dexterity of the tools permits a skilled surgeon to better preserve sexual function nerves with as few as ten percent of patients being impotent as a result.

Although laparoscopic and robotic surgery has shown promising potential in reducing erectile dysfunction as a side effect of prostate gland removal, erectile dysfunction does still occur. In some instances. erectile dysfunction after a laparoscopic or robotic prostatectomy cannot be prevented due to Wallerian degeneration of nerves after even a slight injury. However, in some cases erectile dysfunction results from inadvertent damage done to the neurovascular bundle (NVB) that supports erectile function because the NVB is not where the surgeon expects. Direct visualization and appearance of the presumed NVB has not traditionally been a good indicator of preserved erectile nerves. The NVB travels from the base of the prostate where it joins with the bladder, to the apex (the portion where the urethra enters the penis). These nerves generally travel on opposing sides in a symmetrical fashion on the outside of the prostate capsule on its undersurface at the four and eight o'clock positions, as indicated in FIG. 5 herein. Previous attempts at nerve monitoring during radical prostatectomy used electrical stimulation along the NVB with measurement of the signal in the cavernous bodies of the penis via a measurement of intracavernous pressures, i.e., muscle response (erectile response) to electrical stimulation. This method is not time efficient and recent studies have indicated that outcomes are inconsistent with the intraoperative findings.

### SUMMARY OF THE INVENTION

Therefore a need has arisen for a rapid, efficient and precise system and method which aids in the detection and mapping of nerves during laparoscopic surgery. In accordance with the present invention, systems and methods are provided which substantially reduce the disadvantages and problems associated with previous methods and systems for performing laparoscopic surgery with reduced risk of damage to internal nerves, such as laparoscopic prostate removal with reduced risk of damage to erectile nerves.

More specifically, in a first embodiment of the electrode probe delivery device of this invention, as depicted in FIGS. 1-3, a handle has one or more catheters providing a path for inserting electrode probes (electrodes) through a body and into a body cavity. The electrode probes extend through the catheters and into the body cavity to be accessible for manipulation by a laparoscopic or robotic device. Under the control of a surgeon, the laparoscopic or robotic device pulls the electrode probe(s) into the body cavity for coupling proximate a selected nerve of interest into muscle tissue. An exploratory probe inserted into the body cavity (e.g. the abdomen) through a laparoscopic channel introduces an electric signal into the tissue of a surgery subject proximate the presumed nerve pathway. This signal is received by the electrode probe coupled to the tissue at a distal point proximate the nerve of interest. Because nerve tissue conducts electrical current better than other body tissues, the relative strength of the electrical signal passing through the nerve and recorded at the electrode probe will increase as the exploratory probe is placed closer to the nerve to which the electrode probe is proximately coupled. An analyzer interfaced with the electrode probe provides an indication of the proximity of the exploratory probe to the nerve, via an audible sound or visually-depicted signal that varies in intensity with the intensity of the electrical signal received at the electrode probe. Placement of the exploratory probe at various points along the presumed nerve pathway allows a mapping of the approximate nerve pathway by noting the points at which the electrical signal is relatively strongest. Being able to avoid the approximated nerve pathway while dissecting within the body cavity helps the surgeon to reduce the risk of nerve damage.

In another embodiment of the method, an electrode probe inserted through the body surface via an introducer catheter, cannula or needle and into a body cavity (such as the abdominal cavity, for example) is accessible by a laparoscopic device for placement into muscle tissue proximate a nerve. An exploratory probe placed along a presumed pathway of the nerve introduces an electric current into the tissue surrounding the nerve of interest causing a depolarization of the nerve which results in an action potential. The action potential then propagates along the nerve to the neuromuscular junction (the synapse between the nerve and the muscle cell) where a neurotransmitter (acetylcholine) is released in response to the action potential. This neurotransmitter depolarizes the postsynaptic muscle cells creating an electrical potential received by the electrode probe. Analysis of the electrical signal (electrical potential) supports mapping of the approximate nerve pathway through the body cavity.

The present invention provides a number of important technical advantages. One example of an important technical advantage is that nerve pathways internal to the body are detectable during laparoscopic and robotic surgery to help the surgeon avoid inadvertent damage to the nerves. Detection of nerve pathways is rapid and precise by variable signals. such as an audible tone or visual signal that reflect the proximity of a probe to the nerve pathway. Monitoring of nerve pathways is made possible throughout a surgical procedure with readily accessible probes managed with laparoscopic or robotic tools. Probe pairs provide convenient monitoring where nerves serve an area of interest, such as the pair of NVBs that travel from the four and eight o'clock positions under the prostate gland. Although the first embodiment of a nerve pathway mapping system (shown in FIG. 1) is intended primarily for use with the first embodiment of the electrode probe delivery device (described in further detail below and shown in FIGS. 2-5), if desired, the second embodiment of the electrode probe delivery device (shown in FIGS. 6-10) can also be used in the same general manner of the first mapping system described. Similarly, the first electrode probe delivery device (FIGS. 2-5) can be used in the second mapping system illustrated in FIG. 11.

In a use of the present invention described first hereinbelow, using the system of FIG. 1, the electrical signal applied by the exploratory probe travels through a nerve within a body cavity and is directly received by the electrode probe. Analysis of the strength of the electrical signal supports mapping of the approximate nerve pathway through the body cavity. By having an approximate map of the nerve pathway, the surgeon is able to dissect regions of the body near the nerve with a reduced risk of damage to the nerve. Thus, for instance, cancerous portions of the body are surgically removed by selecting dissection points as far distal to the nerve as is practical.

One example of such a dissection presented below is the dissection of a prostate gland from within the abdominal cavity using laparoscopic or robotic techniques. Identification of the neurovascular bundle (NVB) that supports erectile function helps to preserve those particular nerves that support erectile function after removal of the prostate gland. Repeated probing along the presumed pathway of the NVB proximate to the prostate gland permits a surgeon to map the approximate location of the NVB, allowing the surgeon to select dissection'points that reduce the risk of damage to the NVB. Although a prostate dissection is presented as an exemplary use of the mapping of a nerve pathway through a body cavity, other types of surgeries may benefit from application of the disclosed laparoscopic nerve mapping procedure.

By comparison, and as discussed in detail hereafter, in the next described embodiment of a nerve mapping surgical system, illustrated in FIG. 11, preferably, a dual function probe is used selectively as an exploratory probe or as a transection/cautery tool. The new surgical method embodiment for mapping a nerve also includes an alternate electrode probe delivery device, such as illustrated in FIGS. 6-11. Briefly, in this second embodiment of the mapping system the new electrode probe delivery device permits introduction of electrode wires through the subject's abdominal wall (as in the prostatectomy example). Then the surgeon, using laparoscopic or robotic techniques secures the electrode wires (receivers) into muscle tissue near (proximate) the nerve of interest. The exploratory probe (one aspect of the dual function probe of this system) is then used to introduce an electrical signal to the tissue in the surgical site, surrounding the target nerve, preferably at successive, multiple sites along the presumed nerve pathway, distal to the position of the electrode probe(s). Measurement and analysis of the post-synaptic muscle cell depolarization caused by the propagation of an action potential along the target nerve supports mapping of the nerve pathway to better enable the surgeon to avoid damage to the nerve during the prostatectomy surgery.

The exploratory probe device (20) of the first system nerve mapping surgical system (15) can be used with this second nerve mapping surgical system (150), although not as a cautery device. Furthermore, the exploratory (dual function) probe (200) of the second nerve mapping surgical system (150) can, instead, be used in the first nerve mapping surgical system (15). In addition, both the first and second nerve mapping surgical systems (15, 150) can be used to support mapping of the approximate nerve pathway through the body cavity using either described method of the present invention.

Accordingly, in keeping with the above, the invention is, briefly, an electrode probe delivery device (110). The device includes a base plate (123) having a first side and a second side; an insertion guide (121) having a distal end and a proximal end, the proximal end of the insertion guide being connected to and extending from the first side of the base plate. The distal end of the insertion guide is formed for facile receipt of at least one electrode probe (160). A catheter (140) is extendable longitudinally through the insertion guide and through the aperture of the base plate. The catheter is sufficiently large in diameter to permit receipt of at least one electrode probe. A handle (100) is connected to the catheter and is operable to permit selective movement of the catheter relative to the base plate such that by manipulation of the handle the catheter is longitudinally passable through the base plate for selective introduction into a patient.

The invention is also, briefly, surgical system (150) including at least one electrode probe; an analyzer in operable communication with the at least one electrode probe; and a bipolar instrument (200) for selective use as a exploratory probe and as a surgical instrument. A bipolar power supply (119) is in operable communication with a switch, which is in operable communication with the analyzer, the bipolar power supply and the bipolar instrument to thereby permit an operator of the system to selectively alternate use of the bipolar instrument between use as a surgical instrument and use as an exploratory probe.

The invention is further, briefly, a method of mapping a nerve pathway which includes providing a surgical system (150) having at least one electrode probe, and an optional amplifier in operable communication with the electrode probe. An analyzer of the system is in operable communication with the amplifier and a bipolar instrument (200) for selective use as a exploratory probe and as a surgical instrument. A bipolar power supply of the system is in operable communication with a switch, which is in operable communication with the analyzer, the bipolar power supply and the bipolar instrument, to thereby permit an operator of the system to selectively alternate use of the bipolar instrument between use as a surgical instrument and use as an exploratory probe. The method includes inserting the at least one electrode probe through the surface of the body of the patient and into a body cavity of the patient; using a laparoscopic instrument to selectively position the at least one electrode probe; coupling the at least one electrode probe proximate to the tissue adjacent a selected nerve of interest and inserting the bipolar instrument—exploratory probe (200) into the body cavity through a laparoscopic channel. The method also includes introducing an electric signal into the tissue proximate the presumed nerve pathway with the bipolar instrument—exploratory probe such that the signal passes through the nerve and is received by the at least one electrode probe coupled to the tissue at a distal point proximate the nerve of interest and recording the relative strength of the electrical signal passing through the nerve of interest at the at least one electrode probe, and analyzing the electrical signal recorded, to thereby map the approximate pathway of the nerve of interest based on the relative strength of the electrical signals recorded, then repeatedly placing the exploratory probe at successive points proximate the presumed nerve pathway and repeating the steps of recording and analyzing the electrical signal; and noting the points at which the electrical signal is relatively strongest to thereby map the approximate pathway of the nerve of interest.

Moreover, the invention is, briefly, a method of mapping a nerve pathway including the steps of providing a surgical system having an electrode probe delivery device, attaching the electrode probe delivery device to a surface of a patient's body and inserting at least one electrode probe via the electrode probe delivery device through the surface of the patient's body and into a body cavity of the patient. A laparoscopic instrument is used to position the at least one electrode probe proximate a nerve of interest and an exploratory probe is placed along a presumed pathway of the nerve of interest. An electric current is introduced into tissue surrounding the nerve of interest with the exploratory probe, thereby causing a depolarization of the nerve of interest, resulting in an action potential which propagates along the nerve to the neuromuscular junction and subsequently causes release of neurotransmitter. The method further includes recording an electrical potential received by the at least one electrode probe coupled to the tissue at a distal point proximate the nerve of interest, which electrical potential is created by the neurotransmitter depolarizing postsynaptic muscle cells, and analyzing the electrical potentials recorded to thereby map the approximate pathway of the nerve of interest. The method further includes repeatedly placing the exploratory probe at successive point proximate the presumed nerve pathway and repeating steps or recording and analyzing the electrical potentials and noting the points at which the electrical potential is relatively strongest to thereby map the approximate pathway of the nerve of interest.

### BRIEF DESCRIPTION OF THE DRAWINGS

The present invention may be better understood, and its numerous objects, features and advantages made apparent to those skilled in the art by referencing the accompanying drawings. The use of the same reference number throughout the several figures designates a like or similar element.

FIG. 1 depicts one embodiment of a system for identifying nerve pathways within a body cavity;

FIG. 2 depicts insertion of a dual electrode probe with a catheter through a body surface and into a body cavity;

FIG. 3 depicts positioning of an electrode probe with a laparoscopic device to couple to the body within the body cavity proximate a nerve;

FIG. 4 depicts an exploratory probe providing an electrical current along a presumed pathway of the nerve to map the nerve pathway;

FIG. 5 depicts a postoperative position of the nerve pathway after removal of a prostate gland.

FIG. 6 is a front elevational view of an electrode probe delivery device constructed in accordance with a second embodiment of the present invention.

FIG. 7 is a side elevational view of the electrode probe delivery device of FIG. 6, rotated 90 degrees clockwise on a longitudinal axis.

FIG. 8 is a top plan view of the electrode probe delivery device of FIG. 6.

FIG. 9 is an exploded view of the electrode probe delivery device of FIG. 6, rotated 90 degrees counter-clockwise.

FIG. 10 is a perspective view of the electrode probe delivery device of FIG. 6 laid substantially horizontally, and including two electrode probes protruding from the fully extended catheter of the assembly.

FIG. 11 is a schematic view of a surgical system in keeping with the present invention and including a dual function probe, and being constructed in accordance with and embodying the present invention.

### DETAILED DESCRIPTION

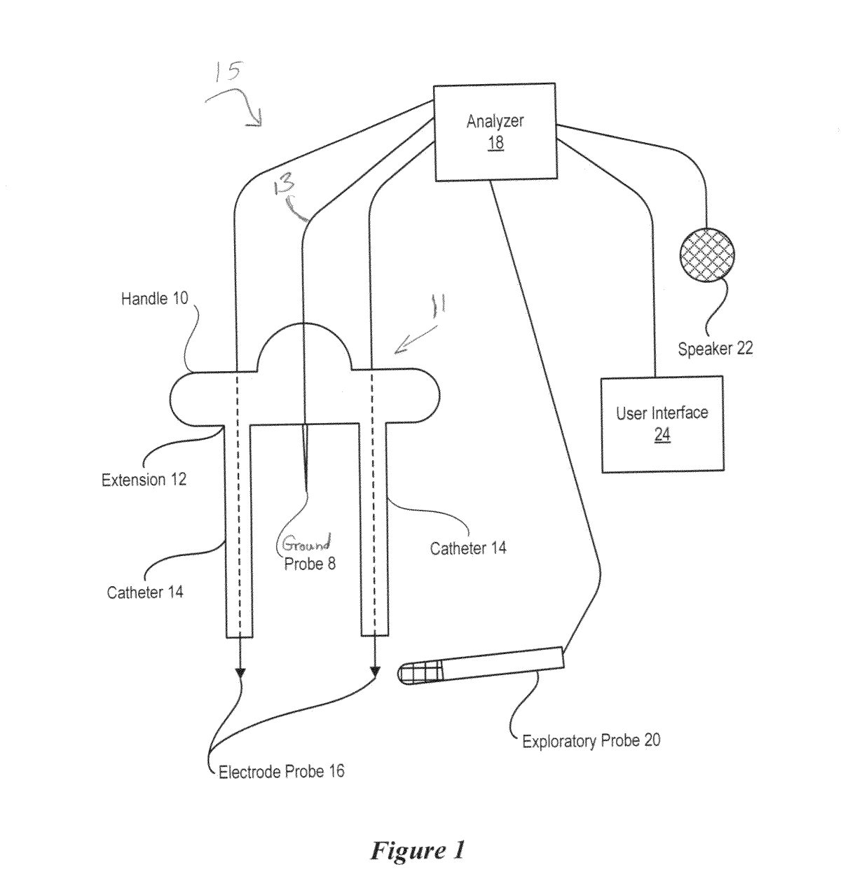

Referring now to FIG. 1, one embodiment of a system, generally designated 15, for identifying nerve pathways within a body cavity is depicted. An electrode probe delivery device 11 has a handle 10, a probe 8 and two extensions 12, each extension having an integrated catheter 14. Handle 10 and catheters 14 provide pathways for travel of electrode probes (probe wires) 16 exposed at the end of each catheter 14. Electrode probes 16 are interfaced through another wire or other communication device (e.g. by radiofrequency transmission) with an analyzer 18 so that electrical signals, such as voltage and current levels, detected at each electrode probe 16 are communicated to analyzer 18. An additional ground probe 8 provides patient grounding when catheters 14 are inserted into the body by interfacing with analyzer 18 through a ground wire 13. Analyzer 18 interfaces with an exploratory probe 20 and selectively sends a predetermined electrical signal (such as a predetermined current) to exploratory probe 20. The electrical signal is passed by exploratory probe 20 to the tissue of a surgery subject proximate a presumed nerve pathway and received by an electrode probe 16 coupled to the body at a distal point also proximate the presumed nerve pathway. Because nerve tissue conducts an electrical current better than other body tissues, the relative strength of the electrical signal passing through the nerve and recorded at an electrode probe 16 will increase as exploratory probe 20 is placed closer to the nerve to which the electrode probe 16 is proximately coupled. Thus, by moving exploratory probe 20 along the presumed nerve pathway, analyzer 18 approximately maps the actual nerve pathway based upon the strength of the signal recorded at electrode probe 16 for each position of exploratory probe 20.

The surgical system depicted by FIG. 1 is built from material adapted for use by laparoscopic and robotic devices to support mapping of nerve pathways through a body cavity. For example, handle 10, catheters 14 and electrode probes 16 are built as an integrated unit (electrode probe delivery device 11) for one time use and disposal. For instance, handle 10 is formed from non-conductive hard plastic and catheter 14 is a large bore stainless steel needles, although other types of catheters or cannulae may suffice as introducers for the electrode probes, such as those made of rigid plastic, such as polycarbonate, for example, or flexible plastic such as polyurethane, for example. Electrode probes 16 are preferably thin, insulated wires which have a length sufficient to reach the desired area within the body cavity when grabbed by a laparoscopic or robotic device and pulled from the end of catheter 14 to the desired area. As an example of dimensions that suffice for a prostatectomy, the length of catheters 14 extending from handle 10 is approximately between 45 mm and 65 mm. Electrode probes 16 are, for example, recording electrodes suitable for manipulation by a laparoscopic robotic arm under direct laparoscopic vision, such as with an extendable length of approximately 50 cm from the end of catheter 14. Analyzer 18 interfaces with a speaker 22, which outputs an auditory signal having an interval and intensity related to the strength of the electrical potential detected at electrode probe 16. Thus, for example, a surgeon maps a nerve pathway by listening to the intensity of the auditory response output at speaker 22 and comparing the auditory responses of different positions of exploratory probe 20. Additionally, a user interface 24 can provide visual analysis of the nerve pathway as well as control over the signal provided at exploratory probe 20.

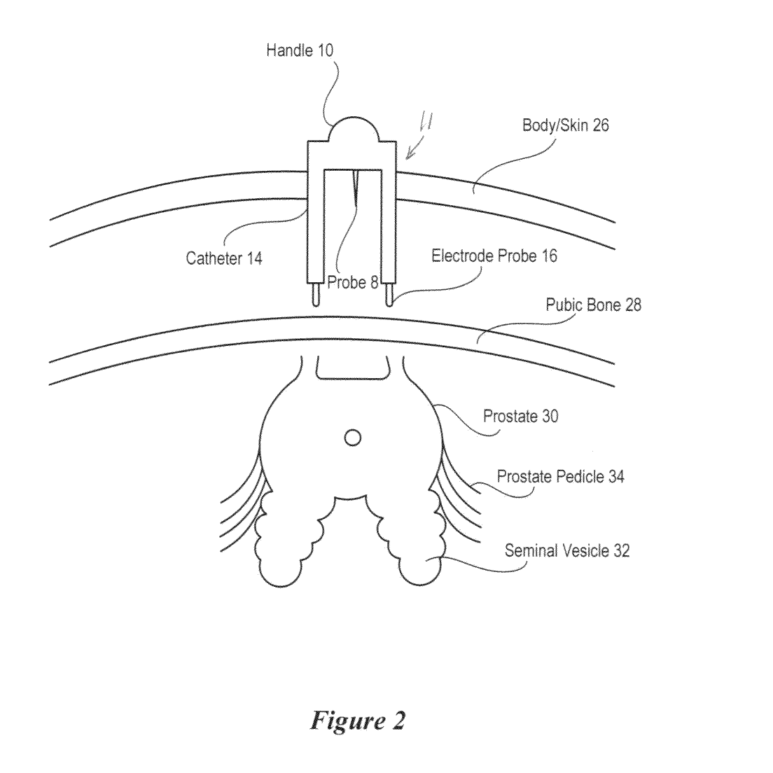

Referring now to FIG. 2, insertion of an electrode probe delivery device 11 with a catheter 14 through a body surface and into a body cavity is depicted. Handle 10 engages the skin of the subject's body to insert catheters 14 into the abdominal cavity suprapubicly. Catheter 14 provides a pathway through abdominal wall 26 to provide access for electrode probes 16 within an abdominal cavity. The pubic bone 28 sits above the prostate 30 and seminal vesicles 32 within the abdominal cavity. Dissection of prostate 30 requires cutting at the prostate pedicle 34, through which neurovascular bundles (NVBs) travel that support erectile function. During laparoscopic or robotic surgery, laparoscopic devices or robotic devices, such as robotic arms, are inserted into the abdominal cavity to allow dissection of prostate 30.

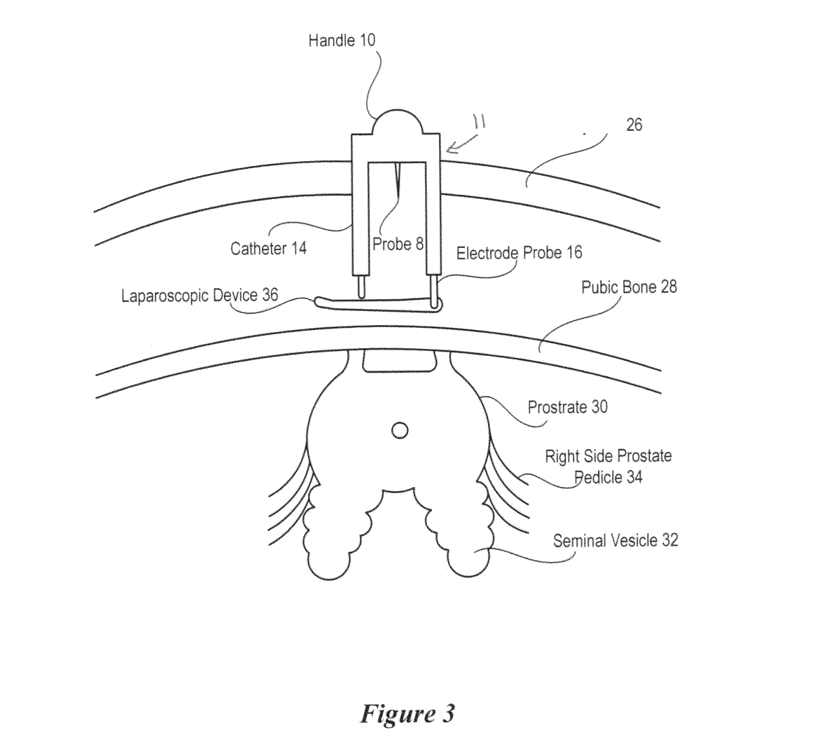

Referring now to FIG. 3, positioning of an electrode probe 16 with a laparoscopic device 36 to couple to the tissue within the body cavity proximate a nerve is depicted. Electrode probe delivery device 11 allows safe insertion of electrode probes 16 where the electrode probes 16 are visible to laparoscopic vision while simultaneously grounding the body through ground probe 8. Under direct laparoscopic vision, the surgeon, using a laparoscopic device or robotic device 36, grabs an electrode probe 16 to move it to a desired location proximate a nerve of interest. Laparoscopic device 36 is used to advance an electrode probe 16 on the right hand side of the figure to a position proximate the NVB on the right hand side of prostate 30, and to advance the other electrode probe 16 on the left hand side of the figure to a position proximate the NVB on the left hand side of prostate 30. Electrode probes 16 temporarily couple to the body so that they remain in position until removed by use of laparoscopic device 36. While the above procedure has been explained using two electrode probes 16 to facilitate the mapping procedure, it will be understood by one skilled in the art that the process can be managed using only one electrode probe 16, or even with more than two such electrode probes.

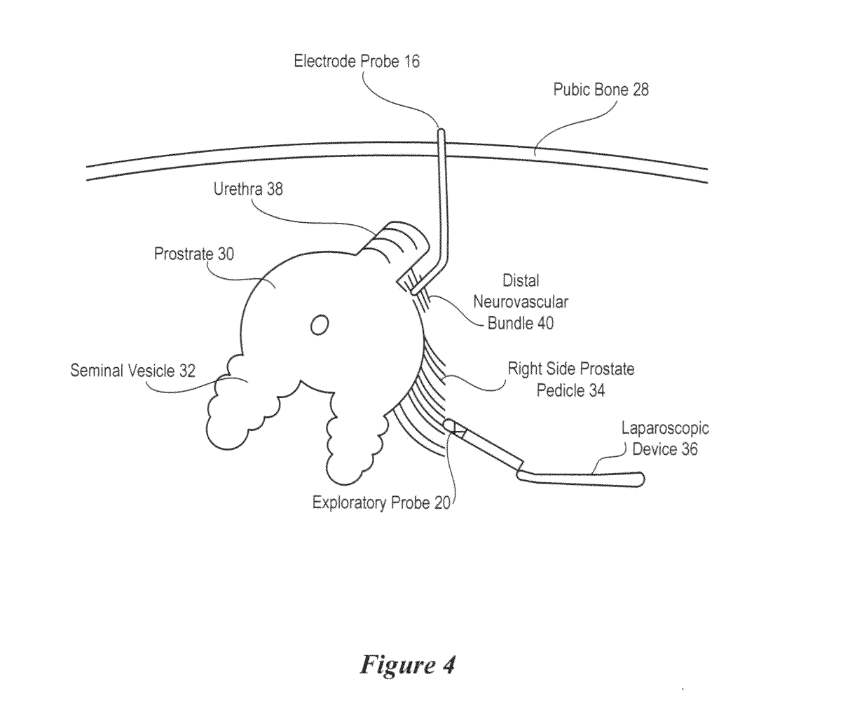

Referring now to FIG. 4, an exploratory probe 20 providing an electrical current to tissue surrounding the nerve of interest to map the nerve pathway is depicted. Electrode probe 16 attaches at the apex of prostate 30 near where the urethra enters the penis. The position at the apex of prostate 30 is a consistent position for the distal NVB 40 just before it exits the pelvis. Electrode probe 16 is of appropriate length to attach at the apex of prostate 30 and is insertable into handle 10 and catheters 14 to make connection of the electrode probe 16 to the body simple and reliable. Once electrode probe 16 is connected to the body tissue near the normal location of the nerve of interest, electrical stimulation is achieved by delivering a low stimulation current (0.5-10 milliamps) through an exploratory probe 20. The nerves of interest (e.g. erectile nerves) are stimulated more or less depending on the proximity of the exploratory probe 20. Initial searching for the nerve is performed at relatively high stimulus intensities, such as up to 3.5 milliamps, or even possibly as high as 10 milliamp of current. Searching is performed by placing exploratory probe 20 at several places around the presumed pathway of the nerve and listening for an auditory response from the speaker 22 as well as a visual confirmation on user interface 24 as provided by analyzer 18 that reflects the strength of the electrical signal received at the electrode probe 16. Once a nerve's general pathway is mapped, the stimulus intensity provided at exploratory probe 20 can be reduced, such as to a level of one-half a milliamp, to map the nerve's location more precisely.

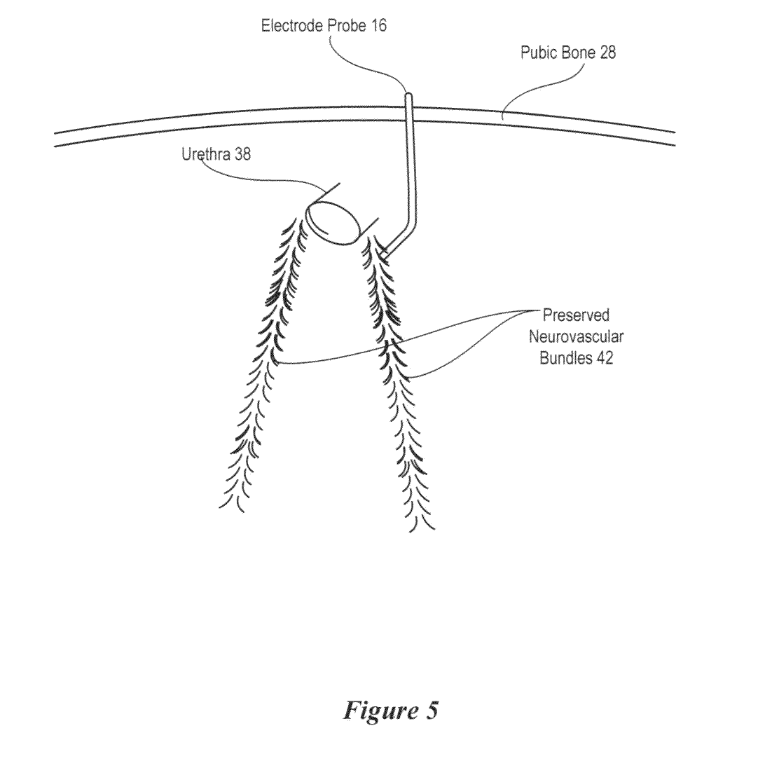

Referring now to FIG. 5, a postoperative position of the nerve pathway after removal of a prostate gland is depicted. Throughout dissection of the prostate, exploratory probe 20 is used, by selectively repositioning it and repeatedly introducing an electrical signal at a predetermined strength, to re-map and confirm the position of NVB 42. The objective is to confirm the location of the erectile nerves so that tissue may be selectively preserved or removed depending on the aggressiveness of the cancer. Although the example presents preservation of the nerves to the NVB during a prostatectomy, other types of nerves may be identified and preserved within the abdominal cavity or other body cavities, depending upon the surgical procedure being performed.

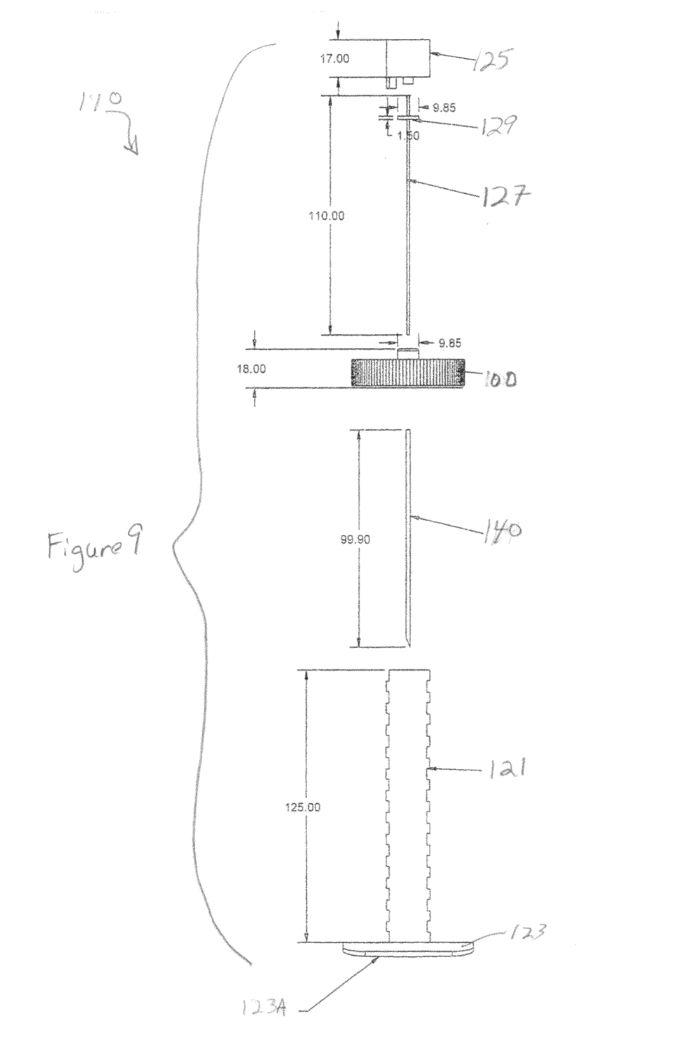

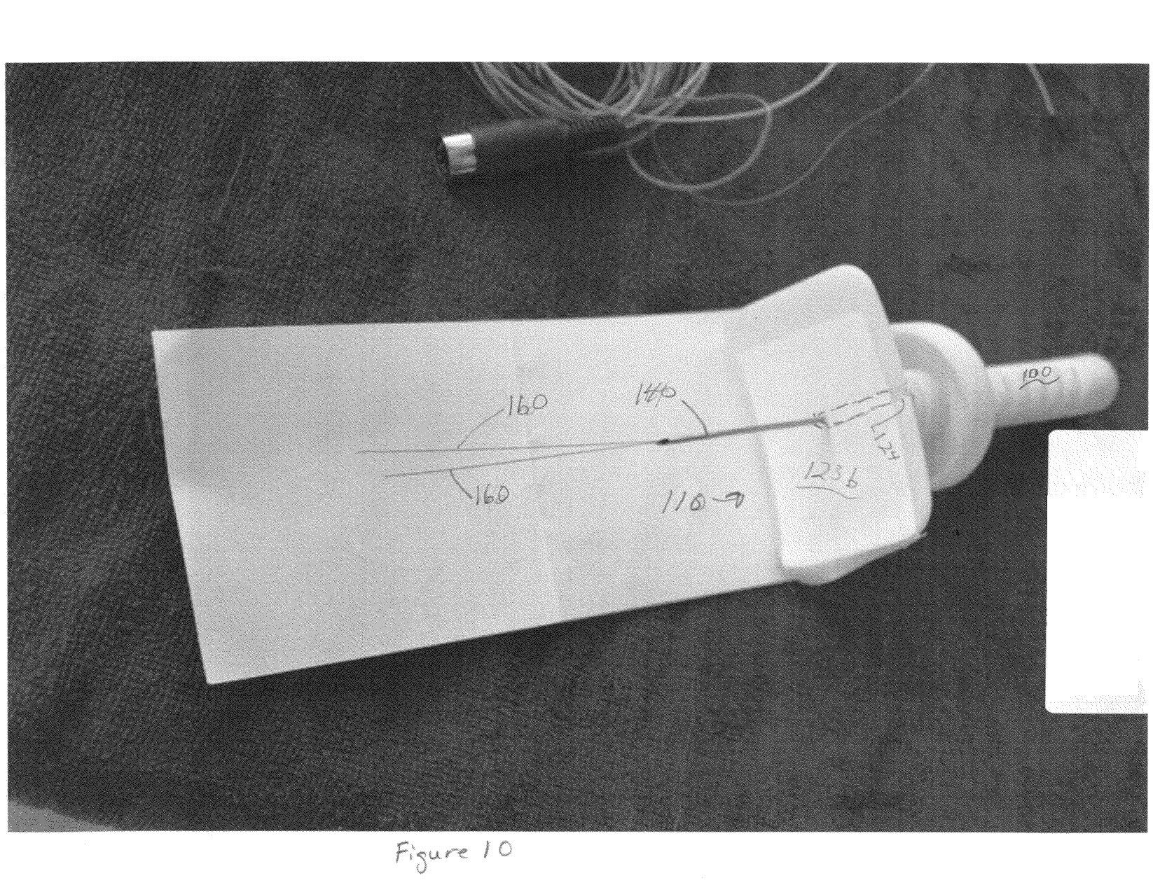

FIGS. 6-10 illustrate a further embodiment of the electrode probe delivery device 110 of the invention for use in alternative system 150 (shown in FIG. 11), wherein a dual function probe 200 is used for both surgery (e.g. transaction and cauterization) and as the exploratory probe in the herein described nerve mapping procedure. The dimensions shown in FIGS. 6-9 are in millimeters and are exemplary of a useful size and shape of the preferred embodiment of electrode probe delivery device 110, although the device is not limited to these dimensions or the specific shape shown.

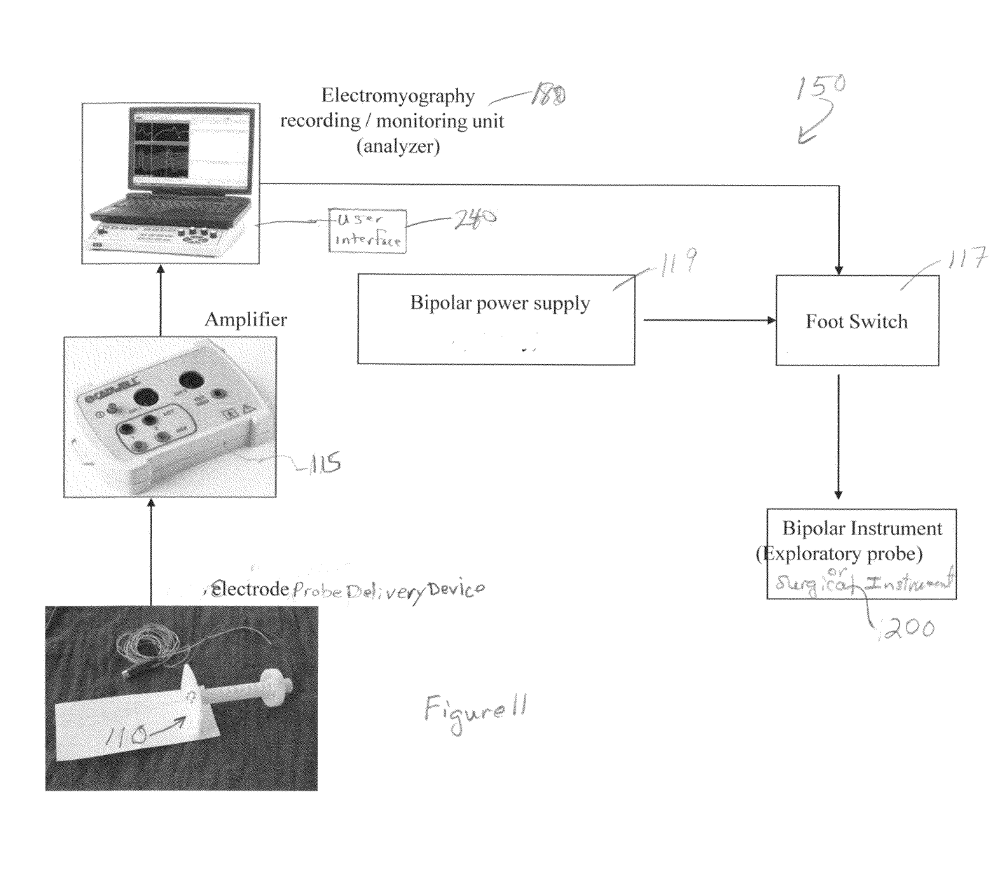

While having some similarities in function, electrode probe delivery device 110 is structured quite differently than the electrode probe delivery device 11 of FIG. 1. In this second embodiment the electrode “introducer” or electrode probe delivery device 110 includes an elongated insertion guide 121 having base plate 123 mounted transversely at one end thereof. An aperture 124, indicated in FIG. 10, is defined by base plate 123 and permits passage through the base plate of catheter 140. The shape of aperture 124 may be annular, groove-like or otherwise shaped, as may be convenient for manufacture and/or use. Base plate 123 is preferably (although not necessarily) provided with a slightly concave curved surface 123A which is positioned downwardly, proximate the patient and usually against the patient's abdominal wall during use, as in the position shown in FIG. 6. Base plate 123 is further preferably provided on its lower, or concave, surface 123A with a coating of a suitable surgical grade adhesive (not shown), which is preferably over-layered with a protective liner 123B until use. The adhesive serves to removably secure electrode probe delivery device 110 to a patient's abdominal wall skin in normal use position, so that insertion guide 121 extends substantially perpendicular from the patient's body surface, such as on the exterior abdomen, for example.

For the most part, electrode probe delivery device 110 is preferably formed of a suitable surgical grade plastic which can be sterilized at least once for optional reuse, and can also, if preferred, be disposed of after a single use. In this preferred embodiment catheter 140 is a large bore steel needle, although other types of catheters or cannulae, such as those made of rigid plastic such as polycarbonate for example or flexible plastic such as polyurethane for example, may suffice as introducers for the probe electrodes. At the uppermost (distal) end of electrode probe delivery device 110 there is an adaptation such as a funnel 125, for example, which is preferably integral to device 110, to readily receive electrode probes 160, which are shown in FIG. 10, extending from catheter 140 of delivery device 110. A section of tubing 127 (e.g. PEEK tubing) is preferably secured to the proximal end of funnel 125 and is preferably secured thereto by a glue plug 129 or other suitable mechanism, such as by friction fit. A catheter 140 longitudinally and slidingly receives tubing 127, so as to form a telescoping tube which can be extended entirely through insertion guide 121 by selective turning and advancing of a preferably knurled handle 100. Alternatively, a useful embodiment of the electrode probe delivery device 110 can be formed with the catheter alone, without the guiding tubing 127.

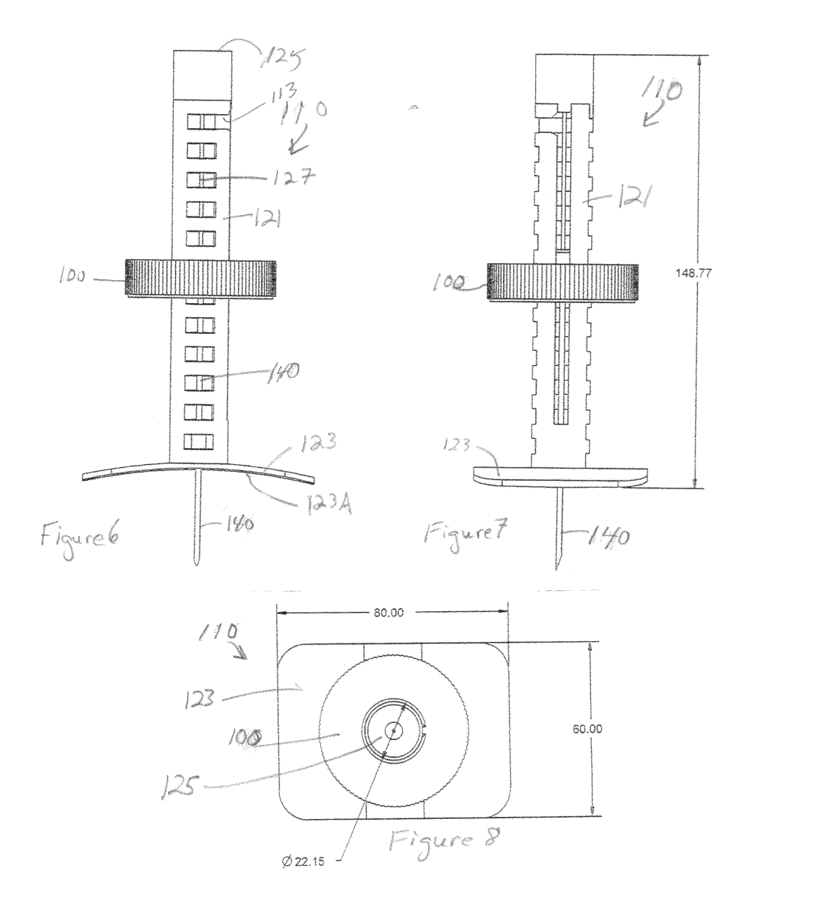

Handle 100 is preferably, although not necessarily, rotatably mounted around the exterior of insertion guide 121. Handle 100 is further preferred to have a simple locking position (acquired, for example, by rotation into groove such as that indicated at 113 in FIG. 6) so that a simple one-handed movement by the surgeon will permit catheter 140 to be slidingly (telescopingly) extended over tubing 127 as the catheter is inserted through the patient's abdominal wall and is selectively positioned by the surgeon under direct laparoscopic vision. FIGS. 6 and 7 show catheter 140 partially extended from insertion guide 121. By contrast, in FIG. 10 catheter 140 is fully extended and the electrode probes 160 (in this case, wires) are seen extending from the open proximal end of catheter 140.

Once electrode assembly 110 is secured to the patient, and catheter 140 is fully extended into the surgical subject's abdomen (for example), electrode probes 160 can be readily inserted through funnel 125 and on through tubing 127 and catheter 140 to a position where probes 160 are visible to laparoscopic vision. Under direct laparoscopic vision, the surgeon, using a laparoscopic device or robotic device, such as element number 36 in FIG. 4, grabs the electrode probes 160 to move probes 160 to desired location proximate a nerve of interest. In similar fashion as in the first-described embodiment, electrode probes 160 are interfaced through a wire or other communication device with an analyzer 180. A grounding probe, not shown in this embodiment provides patient grounding when catheter 140 is inserted into the body by interfacing with analyzer 180 through a ground wire.

Analyzer 180 interfaces with exploratory probe 200 and selectively sends a predetermined electrical signal to exploratory probe 200, such as a predetermined current. The electrical signal is passed by exploratory probe 200 to the body tissue of a surgery subject proximate a presumed nerve pathway. The electric current causes a depolarization of the nerve, which results in an action potential. The action potential then propagates along the nerve to the neuromuscular junction (the synapse between the nerve and the muscle cell) where a neurotransmitter (acetylcholine) is released in response to the action potential. This neurotransmitter depolarizes the postsynaptic muscle cells creating an electrical potential received by the electrode probe 160 coupled to the body tissue at a point distal to the site of the exploratory probe 200 and also proximate the presumed nerve pathway. The relative strength of the electrical potential received at an electrode probe 160, the receiving electrode, will increase as exploratory probe 200 is placed closer to the nerve to which the electrode probe 160 is proximately coupled. Thus by moving exploratory probe 200 along the presumed nerve pathway, analyzer 180 approximately maps the actual nerve pathway based upon the strength of the signal received at electrode probe 160 for each position of exploratory probe 200.

The nerves of interest (e.g. erectile nerves) are stimulated more or less depending upon the proximity of the dual function probe when functioning as an exploratory probe 200. Initial searching for the nerve is performed at relatively high stimulus intensities, such as up to 3.5 milliamp, or even possibly as high as 10 milliamp of current. Searching is performed by placing exploratory probe 200 at several places proximate the presumed pathway of the nerve and listening of an auditory response from a speaker (not shown, but similar to speaker 22 in the first embodiment), as well as a visual confirmation on user interface 240, as provided by analyzer 180 that reflects the strength of the electrical potential received at electrode probe 160. Once the nerve's general pathway is mapped, the stimulus intensity provided at exploratory probe 200 can be reduced, such as to a level of one-half a milliamp, to map the nerve's location more precisely.

Referring to FIG. 11, in this alternative system embodiment, generally designated 150, a different type of laparoscopic or robotic instrument is used as the exploratory probe 200. The preferred instrument is of a known type of laparoscopic or robotic bipolar instrument used for other purposes requiring electric energy transfer during surgery (to transect and cauterize tissue in the case of the laparoscopic or robotic bipolar instruments) and is also used here as an exploratory probe 200. Such a dual function (bipolar instrument/exploratory probe) probe 200 is included in the embodiment of the present system 150. This is an entirely new concept for such nerve mapping/nerve locating procedures, described above, during prostatectomy or other such sensitive surgical procedures. The configuration illustrated in FIG. 11 is the presently preferred embodiment. However, other useful embodiments of the new system 150 can be conceived by the skilled artisan that will still fall within the scope of the invention.

As illustrated in FIG. 11, the electrode probes 160 of the electrode probe delivery device 110 are in operable communication with an optional amplifier 115, which is in turn in operable communication with an electromyography recording/monitoring unit, i.e., analyzer 118. Analyzer 118 is in electronic communication with a switch 117, such as a conventional foot switch, for example, which is operably connected to both a bipolar cautery power supply 119 and a dual function probe 200 in order to facilitate easy and rapid switching by the surgeon between the surgical (transect/cautery) mode of use of dual function probe 200 and the exploratory probe (alternative) function thereof. It should be understood that in this embodiment of the new system 150, it can be possible for the amplifier, analyzer and bipolar power supply to be combined, one or more elements all within the same physical unit or “box” at the discretion of the user. Likewise, in system 15 the analyzer, user interface and power supply can all (or some) be combined in a single unit without departing from the scope of the invention. Further, it is conceived that the amplifier can be replaced with some other mechanism or new technology to adequately effect increase in the signal, if desired. A conventional grounding probe, not shown, is also preferably provided, as in the system described above in relation to the first embodiment of the probe delivery device 11, to attach to the patient for grounding during the entire procedure.

Further with regard to a dual mode of use of dual function probe 200 indicated in FIG. 11, this instrument can function not only as the exploratory probe required to perform nerve mapping as described above, but also as a bipolar cautery surgical instrument used to transect and cauterize tissue during surgery. When used as an exploratory probe, dual function probe 200 produces a constant current single pulse energy output variable between 0-15 mA with 400V compliance voltage, maximum power of 6 watt or less, a variable pulse width of 50-500 microsec (normally 300 microsec) and a variable stimulus repetition rate 1-50/sec (normally 3.5/sec). When operating as a bipolar cauterizing surgical instrument, dual function probe 200 provides a sinusoid high frequency energy output delivering a maximum of 750V (peak to peak) and maximum power of 100 watts. The foot switch 117 indicated in FIG. 11, allows the surgeon to switch the energy going to dual function probe 200 from a constant current single pulse energy sent by the analyzer 180 to a sinusoidal high frequency energy sent by the bipolar cautery power supply 119.

With reference to the discussion above, it should be understood that if desired, electrode probe delivery (insertion) device 11 of the first nerve mapping surgical system embodiment 15 can also be used in the second nerve mapping surgical system embodiment 150. Moreover, the electrode probe delivery device 110 of the second nerve mapping surgical system embodiment 150 can, if preferred, be substituted for delivery device 11 into the first nerve mapping surgical system 15. In addition, both the first and second nerve mapping surgical systems 15, 150 can be used to support mapping of the approximate nerve pathway through the body cavity using either described method of the present invention.

As is readily apparent, new systems 15, 150, including new electrode probe delivery devices 11, 110, respectively, provide efficient and more economical methods for performing surgeries, such as prostatectomies, for example, during which accurate location of critical nerves is desired. Although the present invention has been described in detail, it should be understood that various changes, substitutions and alterations can be made hereto without departing from the spirit and scope of the invention as defined by the appended claims.

The above description of the preferred embodiments is merely exemplary in nature and is in no way intended to limit the invention, its application, or uses.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An electrode probe delivery device (110) comprising:
a base plate (123) having a first side and a second side;
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, said catheter being sufficiently large in diameter to permit receipt of at least one electrode probe; and
a handle (100) connected to the catheter and operable to permit selective movement of the catheter relative to the base plate such that by manipulation of the handle the catheter is longitudinally passable through the base plate for selective introduction into a patient.

2. The electrode probe delivery device of claim 1, wherein the insertion guide is elongated and substantially tubular.

3. The electrode probe delivery device of claim 1, wherein the base plate second side has a surface (123A) which is slightly concave to thereby enhance the fit of the base plate against the surface of the patient's body.

4. The electrode probe delivery device of claim 3, wherein the base plate second side surface has an adhesive coating and a protective liner over-layering the slightly concave surface, which protective liner is readily removable prior to placement of the electrode probe delivery device in normal operative position against the skin of a patient, to thereby facilitate holding of said electrode probe delivery device in normal use position.

5. The electrode probe delivery device of claim 1, wherein said handle (100) is annular and said catheter is mounted centrally thereto.

6. The electrode probe delivery device of claim 2, and further comprising a tube disposed coaxially within the insertion guide and having an interior diameter sufficiently large to receive and permit passage therethrough of the at least one electrode probe, the tube further having an exterior diameter sufficiently small to permit telescoping receipt of the tube interior through the catheter.

7. The electrode probe delivery device of claim 5, wherein the distal end of said insertion guide is formed to permit selective interlocking engagement with said handle, such that rotational movement of said handle releases said handle from the interlocked engagement and permits said handle to be selective moved longitudinally and proximally, toward said base plate to thereby cause proximal movement of said catheter to and through the skin of a patient to permit introduction of at least one electrode probe through the catheter into a body cavity of the patient to a selectable position.

8. The electrode probe delivery device of claim 1, wherein said electrode probe delivery device is formed at least in part of surgical grade plastic suitable for sterilization and reuse.

9. The electrode probe delivery device of claim 1, wherein said electrode probe delivery device is formed at least in part of stainless steel.

10. The electrode probe delivery device of claim 1, wherein the base plate defines an aperture (124).

11. An apparatus consisting of the combination of an electrode probe delivery device and at least one electrode probe, wherein the electrode probe delivery device comprises a base plate (123) having a first side and a second side;
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, said catheter being sufficiently large in diameter to permit receipt of at least one electrode probe; and
a handle (100) connected to the catheter and operable to permit selective movement of the catheter relative to the base plate such that by manipulation of the handle the catheter is longitudinally passable through the base plate for selective introduction into a patient.

12. The combination of claim 11, wherein the at least one electrode probe (160) comprises two electrode wires.

13. The combination of claim 11, wherein the base plate (123) defines an aperture (124).

14. A surgical system (150) comprising:
at least one electrode probe;
an analyzer in operable communication with the at least one electrode probe;
a bipolar instrument (200) for selective use as a exploratory probe and as a surgical instrument;
a bipolar power supply (119) in operable communication with a switch; and
a switch in operable communication with said analyzer, said bipolar power supply and said bipolar instrument, to thereby permit an operator of said system to selectively alternate use of said bipolar instrument between use as a surgical instrument and use as an exploratory probe.

15. The surgical system of claim 14, and further comprising a user interface in operable communication with the analyzer.

16. The surgical system of claim 14, and further comprising an amplifier in operable communication with the analyzer.

17. The combination of a surgical system (150) and an electrode probe delivery device, wherein the surgical system comprises:
at least one electrode probe;
an analyzer in operable communication with the at least one electrode probe;
a bipolar instrument (200) for selective use as a exploratory probe and as a surgical instrument;
a bipolar power supply (119) in operable communication with a switch; and
a switch in operable communication with the analyzer, the bipolar power supply and the bipolar instrument, to thereby permit an operator of said system to selectively alternate use of the bipolar instrument between use as a surgical instrument and use as an exploratory probe.

18. The surgical system of claim 17, and further comprising an amplifier in operable communication with the analyzer.

19. The surgical system of claim 17, and further comprising a user interface in operable communication with the analyzer.

20. The surgical system of claim 17, wherein the electrode probe delivery device (110) comprises: a base plate (123) having a first side and a second side, said base plate defining an aperture (124);
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, said catheter being sufficiently large in diameter to permit receipt of at least one electrode probe; and
a handle (100) connected to the catheter and operable to permit selective movement of the catheter relative to the base plate such that by manipulation of the handle the catheter is longitudinally passable through the base plate for selective introduction into a patient.

21. The surgical system of claim 14, wherein the switch is a foot switch.

22. The surgical system of claim 14, and further comprising an electrode probe delivery device, wherein the electrode probe delivery device (11) comprises:
a handle,
at least one catheter coupled to the handle, the at least one catheter being operable to insert into a surface of the body of a patient to provide access to the body cavity of the patient; and wherein
the electrode probe is adapted to extend through the at least one catheter to a position accessible by a laparoscopic device disposed in the body, the electrode probe operable to further extend from the catheter, under the influence of a laparoscopic device, to couple to the body within the body cavity proximate to a nerve.

23. A method of mapping a nerve pathway comprising:
a) providing a surgical system (150) having at least one electrode probe; an analyzer in operable communication with the electrode probe; a bipolar instrument (200) for selective use as a exploratory probe and as a surgical instrument; a bipolar power supply in operable communication with a switch; and a switch in operable communication with the analyzer, the bipolar power supply and the bipolar instrument, to thereby permit an operator of the system to selectively alternate use of the bipolar instrument between use as a surgical instrument and use as an exploratory probe; inserting the at least one electrode probe through the surface of the body of the patient and into a body cavity of the patient;
b) using a laparoscopic instrument to selectively position the at least one electrode probe;
c) coupling the at least one electrode probe proximate to the tissue adjacent a selected nerve of interest;
d) inserting the bipolar instrument—exploratory probe (200) into the body cavity through a laparoscopic channel;
e) introducing an electric signal into the tissue proximate the presumed nerve pathway with the bipolar instrument—exploratory probe such that the signal passes through the nerve and is received by the at least one electrode probe coupled to the tissue at a distal point proximate the nerve of interest;
f) recording the relative strength of the electrical signal passing through the nerve of interest at the at least one electrode probe;
g) analyzing the electrical signal recorded at step f) to thereby map the approximate pathway of the nerve of interest based on the relative strength of the electrical signals recorded;
h) repeatedly placing the exploratory probe at successive points proximate the presumed nerve pathway and repeating steps f) and g); and
i) noting the points at which the electrical signal is relatively strongest to thereby map the approximate pathway of the nerve of interest.

24. The method of claim 23, wherein step a) includes providing a user interface in operable communication with the analyzer.

25. The method of claim 23, wherein step a) includes providing an amplifier in operable communication with the analyzer.

26. The method of claim 23, wherein step a) includes providing an electrode probe delivery device and further comprising, between steps a) and b) the step of attaching the electrode probe delivery device to the surface of the body of a patient.

27. The method of claim 26, wherein step a) includes providing an electrode probe delivery device (110) having a base plate (123) having a first side and a second side and defining an aperture (124);
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, said catheter being sufficiently large in diameter to permit receipt of at least one electrode probe; and
a handle (100) connected to the catheter and operable to permit selective movement of the catheter relative to the base plate such that by manipulation of the handle the catheter is longitudinally passable through the base plate for selective introduction into a patient.

28. The method of claim 26, wherein step a) includes providing an electrode probe delivery device (11) having a handle, at least one catheter coupled to the handle, the at least one catheter being operable to insert into a surface of the body of a patient to provide access to the body cavity of the patient; and wherein the at least one electrode probe is adapted to extend through the at least one catheter to a position accessible by a laparoscopic device disposed in the body, the at least one electrode probe operable to further extend from the catheter, under the influence of a laparoscopic device, to couple to the body within the body cavity proximate to a nerve.

29. The method of claim 23, wherein step a) includes:
providing a surgical system (15) for mapping the location of an internal nerve within a body cavity of a body, the system having:
at least one electrode probe adapted for inserting into the body cavity through a surface of the body to a position accessible by a laparoscopic device disposed in the body cavity, the at least one electrode probe being positionable by the laparoscopic device within the body cavity and operable to couple to the body within the body cavity proximate to a preselected nerve; at least one exploratory probe adapted to be disposed in the body cavity and operable to introduce an electrical signal to the body within the body cavity along the presumed pathway of the preselected nerve and distal to the at least one electrode probe to thereby selectively provide an electric signal to the at least one electrode probe; and an analyzer interfaced with the at least one electrode probe, the analyzer being operable to indicate the proximity of the at least one exploratory probe to the preselected nerve based on a measurement of the strength of the electrical signal sensed by the at least one electrode probe and to thereby permit mapping of the location of the preselected nerve.

30. The method of claim 29, and further wherein
step a) includes providing an electrode probe delivery device (110) having a base plate (123) having a first side and a second side and defining an aperture (124);
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, said catheter being sufficiently large in diameter to permit receipt of at least one electrode probe; and
a handle (100) connected to the catheter and operable to permit selective movement of the catheter relative to the base plate such that by manipulation of the handle the catheter is longitudinally passable through the base plate for selective introduction into a patient.

31. A method of mapping a nerve pathway comprising:
a) providing a surgical system having an electrode probe delivery device.
b) attaching the electrode probe delivery device to a surface of a patient's body;
c) inserting at least one electrode probe via the electrode probe delivery device through the surface of the patient's body and into a body cavity of the patient;
d) using a laparoscopic instrument to position the at least one electrode probe proximate a nerve of interest;
e) placing an exploratory probe along a presumed pathway of the nerve of interest;
f) introducing an electric current into tissue surrounding the nerve of interest with the exploratory probe and thereby causing a depolarization of the nerve of interest, resulting in an action potential which propagates along the nerve to the neuromuscular junction and subsequently causes release of neurotransmitter;
g) recording an electrical potential received by the at least one electrode probe coupled to the tissue at a distal point proximate the nerve of interest, which electrical potential is created by the neurotransmitter depolarizing postsynaptic muscle cells; and
h) analyzing the electrical potentials recorded at step g) to thereby map the approximate pathway of the nerve of interest;
i) repeatedly placing the exploratory probe at successive point proximate the presumed nerve pathway and repeating steps g) and h); and
j) noting the points at which the electrical potential is relatively strongest to thereby map the approximate pathway of the nerve of interest.

32. The method of claim 31, wherein step a) includes providing a system (15) also having an exploratory probe (20), a user interface (24), at least one of a speaker (22) and a visual monitor, and an analyzer (18), wherein the analyzer is in operative communication with each of the electrode probe delivery device, the exploratory probe, the user interface, and the at least one of a speaker and a visual monitor.

33. The method of claim 32, wherein step a) includes providing an electrode probe delivery device (11) having a handle, at least one catheter coupled to the handle, the at least one catheter being operable to insert into a surface of the body of a patient to provide access to the body cavity of the patient; wherein the electrode probe is adapted to extend through the at least one catheter to a position accessible by a laparoscopic device disposed in the body, the electrode probe operable to further extend from the catheter, under the influence of a laparoscopic device, to couple to the body within the body cavity proximate to a nerve.

34. The method of claim 32, wherein step a) includes an electrode probe delivery device (110) having
a base plate (123) having a first side and a second side and defining an aperture (124);
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, catheter being sufficiently large in diameter to permit receipt of at least one electrode probe; and
a handle (100) connected to the catheter and operable to permit selective movement of the catheter relative to the base plate such that by manipulation of the handle the catheter is longitudinally passable through the base plate for selective introduction into a patient.

35. The method of claim 31, wherein step a) includes providing a system (150) also having:
an analyzer in operable communication with the at least one electrode probe;
a bipolar instrument (200) for selective use as a exploratory probe and as a surgical instrument:
a bipolar power supply in operable communication with a switch; and
a switch in operable communication with the analyzer, the bipolar power supply and the bipolar instrument, to thereby permit an operator of the system to selectively alternate use of the bipolar instrument between use as a surgical instrument and use as an exploratory probe.

36. The method of claim 31, wherein step a) includes providing an amplifier in operable communication with the analyzer.

37. The method of claim 31, wherein step a) includes providing an electrode probe delivery device (11) having a handle, at least one catheter coupled to the handle, the at least one catheter being operable to insert into a surface of the body of a patient to provide access to the body cavity of the patient; wherein the electrode probe is adapted to extend through the at least one catheter to a position accessible by a laparoscopic device disposed in the body, the electrode probe operable to further extend from the catheter, under the influence of a laparoscopic device, to couple to the body within the body cavity proximate to a nerve.

38. The method of claim 31, wherein step a) includes providing an electrode probe delivery device (110) having a base plate (123) having a first side and a second side and defining an aperture (124) for passage there through of the at least one electrode probe:
an insertion guide (121) having a distal end and a proximal end, the proximal end of the insertion guide being connected to and extending from the first side of the base plate, and the distal end of the insertion guide being formed for facile receipt of at least one electrode probe (160);
a catheter (140) extendable longitudinally through the insertion guide and through the aperture of the base plate, the catheter being sufficiently large in diameter to permit receipt of the at least one electrode probe; and
a handle (100) connected to the catheter and operable to permit selective movement of the catheter relative to the base plate such that by manipulation of the handle the catheter is longitudinally passable through the base plate for selective introduction into a patient.

39. A surgical system for mapping the location of an internal nerve within a body cavity, the system comprising:
at least one electrode probe adapted for inserting into the body cavity through a surface of the body to position accessible by a laparoscopic device disposed in the body cavity, the at least one on electrode probe being positionable by the laparoscopic device within the body cavity and operable to couple to the body within the body cavity proximate to a preselected nerve; at least one exploratory probe adapted to be disposed in the body cavity and operable to introduce an electrical signal to the body within the body cavity along the presumed pathway of the preselected nerve and distal to the at least one electrode probe to thereby selectively provide an electric signal to the at least one electrode probe; and an analyzer interfaced with the at least one electrode probe, the analyzer being operable to indicate the proximity of the at least one exploratory probe to the preselected nerve based on a measurement of the strength of the electrical signal sensed by the at least one electrode probe and to thereby permit mapping of the location of the preselected nerve; and
further comprising an electrode probe delivery device (110) having a base plate (123) having a first side and a second side; an insertion guide (121) having a distal end and a proximal end, the proximal end of the insertion guide being connected to and extending from the first side of the base plate, and the distal end of the insertion guide being formed for facile receipt of at least one electrode probe (160): a catheter (140) extendable longitudinally through the insertion guide and through the aperture of the base plate, the catheter being sufficiently large in diameter to permit receipt of the at least one electrode probe; and a handle (100) connected to the catheter and operable to permit selective movement of the catheter relative to the base plate such that by manipulation of the handle the catheter is longitudinally passable through the base plate for selective introduction into a patient.

40. The system of claim 39, wherein the base plate defines an aperture (124) for passage there through of the at least one electrode probe.

41. The system of claim 39, wherein the second side surface is slightly concave to thereby enhance the fit of the base plate against the surface of the patient's body.
